(19) United States
(12) Patent Application Publication
(10) Pub. No.: US 2026/0364579 A1
(43) Pub. Date: Nov. 19, 2026

(54) MAKING CATALYSTS FOR OXIDATIVE DEHYDROGENATION

(71) Applicant: NOVA CHEMICALS (INTERNATIONAL) S.A., Friboung (CH)

(72) Inventors: David SULLIVAN, Calgary (CA); Ce YANG, Clarksville, MD (US); Vasily SIMANZHENKOV, Calgary (CA)

(73) Assignee: NOVA CHEMICALS (INTERNATIONAL) S.A., Friboung (CH)

(21) Appl. No.: 19/126,574

(22) PCT Filed: Oct. 20, 2023
(86) PCT No.: PCT/IB2023/060633

§ 371 Date: May 1, 2025

Related U.S. Application Data
(60) Provisional application No. 63/422,097, filed on Nov. 3, 2022.

(51) Int. Cl. B01J 27/057; B01J 21/04; B01J 21/06; B01J 23/00; B01J 37/03; B01J 37/10; C07C 5/48

(52) U.S. Cl. CPC B01J 27/0576; B01J 21/04; B01J 21/063; B01J 23/002; B01J 37/035; B01J 37/10; C07C 5/48; B01J 2523/55; B01J 2523/57; B01J 2523/64; B01J 2523/68; C07C 2523/32

(57) ABSTRACT

Methods are provided for preparing a catalyst for oxidative dehydrogenation of ethane. An exemplary method includes forming a slurry including oxides of molybdenum, tantalum, vanadium, and tellurium. Citric acid, oxalic acid, and ethylene glycol are added to the slurry. A chemically compatible carrier (CCC) is added to the slurry. The slurry is transferred to a hydrothermal synthesis vessel, and the hydrothermal synthesis vessel is heated to form a catalyst material precursor. The catalyst material precursor formed in the hydrothermal synthesis vessel is isolated and calcined to form the catalyst material.

### TECHNICAL FIELD

The present disclosure relates generally to catalyst materials and systems for oxidative dehydrogenation (ODH). More specifically, the catalyst material contains molybdenum (Mo), vanadium (V), tellurium (Te), tantalum (Ta), and oxygen (O), as well as a chemically compatible carrier (CCC).

### BACKGROUND ART

Olefins like ethylene, propylene, and butylene, are basic building blocks for a variety of commercially valuable polymers. Since naturally occurring sources of olefins do not exist in commercial quantities, polymer producers rely on methods for converting the more abundant lower alkanes into olefins. The method of choice for today's commercial scale producers is steam cracking, a highly endothermic process where steam-diluted alkanes are subjected very briefly to a temperature of at least 800 °C. The fuel demand to produce the required temperatures and the need for equipment that can withstand that temperature add significantly to the overall cost. In addition, the high temperature promotes the formation of coke, which accumulates within the system, resulting in the need for costly periodic reactor shutdowns for maintenance and coke removal.

Selective oxidation processes, such as oxidative dehydrogenation (ODH), are an alternative to steam cracking that are exothermic and produce little or no coke. In ODH, a lower alkane, such as ethane, is mixed with oxygen in the presence of a catalyst and optionally a diluent, such as carbon dioxide or nitrogen or steam, which may be performed at temperatures as low as 300 °C, to produce the corresponding alkene. Various other oxidation products may be produced in this process, including carbon dioxide and acetic acid, among others. ODH suffers from lower conversion rates when compared to steam cracking, a fact that when combined with lower selectivity may have prevented ODH from achieving widespread commercial implementation. There is a need for a catalyst material for an ODH of ethane process with high ethylene selectivity, activity, and longevity.

### SUMMARY OF INVENTION

An embodiment described in examples herein provides a method for preparing a catalyst material. The method includes forming a slurry including oxides of molybdenum, tantalum, vanadium, and tellurium. Citric acid, oxalic acid, and ethylene glycol are added to the slurry. A chemically compatible carrier (CCC) is added to the slurry. The slurry is transferred to a hydrothermal synthesis vessel, and the hydrothermal synthesis vessel is heated to form a catalyst material precursor. The catalyst material precursor formed in the hydrothermal synthesis vessel is isolated and calcined to form the catalyst material.

In an aspect, the CCC comprises α-alumina, titania, or WO₃—ZrO₂. In an aspect, the CCC is α-alumina. In an aspect, the CCC is titania. In an aspect, the CCC is WO₃—ZrO₂.

In an aspect, the catalyst material has an ethylene selectivity at 25% ethane conversion within about 5% of the ethylene selectivity of the same catalyst material prepared without the CCC.

In an aspect, anhydrous tantalum oxide is used as the oxide tantalum in the slurry.

In an aspect, the hydrothermal synthesis vessel is heated at 190 °C for 48 hours.

In an aspect, the catalyst material precursor is calcined by placing the catalyst material precursor in a furnace under a purified nitrogen atmosphere, ramping a temperature of the furnace from ambient to a temperature of 600 °C over 6 hours, and holding the temperature at 600 °C for 2 hours.

In an aspect, the catalyst material includes a catalyst having the formula: MoₐV_{b}Te_{c}Ta_{d}Oₓ, wherein a is 1.0, b is about 0.01 to about 0.3, c is about 0.01 to about 0.09, d is about 0.01 to about 0.10, and x is the number of oxygen atoms necessary to render the catalyst electronically neutral.

In an aspect, b is about 0.2 to about 0.4, c is about 0.03 to about 0.07, and d is about 0.03 to about 0.05.

In an aspect, the catalyst has the formula: Mo₁V_{0.3}Te_{0.05}Ta_{0.05}Oₓ.

In an aspect, the catalyst material has a temperature of 25% ethane conversion of from about 323 °C to about 337 °C and a selectivity at 25% ethane conversion of about 97%. In an aspect, the catalyst material has a temperature of 25% ethane conversion of about 323 °C. In an aspect, the catalyst material has a temperature of 25% ethane conversion of about 337 °C.

Another embodiment described in examples provides a process for oxidative dehydrogenation of ethane, the process including contacting a gaseous feed including ethane and oxygen with a catalyst material in a reactor to produce an effluent including ethylene, wherein the catalyst material comprises a catalyst and a chemically compatible carrier (CCC), wherein the catalyst has the formula: MoₐV_{b}Te_{c}Ta_{d}Oₓ. In this formula, a is 1.0, b is about 0.01 to about 0.3, c is about 0.01 to about 0.09, d is about 0.01 to about 0.10, and x is the number of oxygen atoms necessary to render the catalyst electronically neutral. The catalyst material is prepared by forming a slurry including oxides of molybdenum, tantalum, vanadium, and tellurium. Citric acid, oxalic acid, and ethylene glycol are added to the slurry. A chemically compatible carrier (CCC) is added to the slurry. The slurry is transferred to a hydrothermal synthesis vessel, and the hydrothermal synthesis vessel is heated to form a catalyst material precursor. The catalyst material precursor formed in the hydrothermal synthesis vessel is isolated and calcined to form the catalyst material.

In an aspect, the CCC comprises α-alumina, titania, or WO₃—ZrO₂. In an aspect, the CCC is α-alumina. In an aspect, the CCC is titania. In an aspect, the CCC is WO₃—ZrO₂.

In an aspect, the catalyst material has a selectivity at 25% ethane conversion within about 5% of the selectivity of the same catalyst material prepared without the CCC.

In an aspect, b is about 0.2 to about 0.4, c is about 0.03 to about 0.07, and d is about 0.03 to about 0.05. In an aspect, the catalyst has the formula: Mo₁V_{0.3} Te_{0.05}Ta_{0.05}Oₓ. In an aspect, the catalyst has the formula: Mo₁V_{0.3}Te_{0.05} Ta_{0.05}Oₓ.

In an aspect, the catalyst material has a temperature of 25% ethane conversion of from about 323 °C to about 337 °C and a selectivity at 25% ethane conversion of about 97%. In an aspect, the catalyst material has a temperature of 25% ethane conversion of about 323 °C. In an aspect, the catalyst material has a temperature of 25% ethane conversion of about 337 °C.

### BRIEF DESCRIPTION OF THE FIGURES

FIG. 1 is an autoclave used for preparing catalyst materials.

FIGS. 2A and 2B show a test apparatus for testing the ODH performance of the catalyst materials.

FIG. 3 is a plot of the conversion of reagents to ethylene for comparative sample 1C.

FIG. 4 is a plot of the selectivity of ethylene formation for comparative sample 1C.

FIG. 5 is a plot of the conversion/space yields for comparative sample 1C.

FIG. 6 is a plot of the conversion of reagents to ethylene for sample 2E.

FIG. 7 is a plot of the selectivity of ethylene formation for sample 2E.

FIG. 8 is a plot of the conversion/space yields for sample 2E.

FIG. 9 is a plot of the conversion of reagents to ethylene for sample 3E.

FIG. 10 is a plot of the selectivity of ethylene formation for sample 3E.

FIG. 11 is a plot of the conversion/space yields for sample 3E.

FIG. 12 is a plot of the conversion of reagents to ethylene for comparative sample 4C.

FIG. 13 is a plot of the selectivity of ethylene formation for comparative sample 4C.

FIG. 14 is a plot of the conversion/space yields for comparative sample 4C.

FIG. 15 is a plot of the conversion of reagents to ethylene for sample 5E.

FIG. 16 is a plot of the selectivity of ethylene formation for sample 5E.

FIG. 17 is a plot of the conversion of reagents to ethylene for comparative sample 6C.

FIG. 18 is a plot of the selectivity of ethylene formation for comparative sample 6C.

FIG. 19 is a plot of the conversion of reagents to ethylene for comparative sample 6C.

FIG. 20 is a plot of the selectivity of ethylene formation for comparative sample 6C.

FIG. 21 is an XRD graph of sample 1C.

FIG. 22 is an XRD graph of sample 2E.

FIG. 23 is an XRD graph of sample 3E.

FIG. 24 is an XRD graph of sample 4C.

### DESCRIPTION OF EMBODIMENTS

Selective oxidation (SO) is generally used in oxidative dehydrogenation (ODH) reactions to form ethylene, or other alpha-olefins, from ethane. Embodiments described herein provide a synthesis method for a catalyst material including a MoVTaTe catalyst, for example, for the ODH process. In the method, catalyst material precursor powders are mixed, for example, by being ground together, and then used in a hydrothermal synthesis process to form the catalyst materials. In the method, the catalyst material precursor powders include a chemically compatible carrier (CCC). A carrier offers the opportunity to improve the mechanical strength and heat-dissipating properties of the shaped catalyst, increase the accessibility of catalyst active centers to the reactants, and reduce the cost of the catalyst. Previous procedures required multiple steps of the catalyst material synthesis process, such as wet or dry compounding, drying, or second calcining, to incorporate a carrier into the catalyst materials.

However, the procedure comprising multiple steps adds significant costs, energy intensity, and waste. As described herein, it has been determined that a chemically compatible carrier (CCC) could be used directly in the catalyst synthesis process in a single hydrothermal synthesis step.

In addition to a simpler procedure, in embodiments of the methods described herein, the CCC can be added to the catalyst precursors during the hydrothermal synthesis step without significant negative impact to the formation of the active catalyst. For example, the catalyst material showed similar activity and ethylene selectivity to catalyst materials that either lacked carrier or were mechanically mixed with carrier.

Provided herein is a method for preparing a catalyst material that includes forming a slurry including oxides of molybdenum, tantalum, vanadium, and tellurium. Citric acid, oxalic acid, and ethylene glycol are added to the slurry. A chemically compatible carrier (CCC) is added to the slurry. The slurry is transferred to a hydrothermal synthesis vessel, and the hydrothermal synthesis vessel is heated to form a catalyst material precursor. The catalyst material precursor formed in the hydrothermal synthesis vessel is isolated and calcined to form the catalyst material.

In some embodiments, the CCC includes a metal oxide. In some embodiments, the CCC includes α-alumina (α-Al₂O₃), titania (TiO₂), or WO₃—ZrO₂. In some embodiments, the CCC includes α-alumina or titania. In some embodiments, the CCC is α-alumina. In some embodiments, the CCC is titania. In some embodiments, the CCC is WO₃—ZrO₂.

Also provided herein is a process for the oxidative dehydrogenation of ethane, the process including contacting a gaseous feed including ethane and oxygen with a catalyst material in a reactor to produce an effluent including ethylene. The oxidative dehydrogenation catalyst material that includes a catalyst and a chemically compatible carrier (CCC). The catalyst includes molybdenum (Mo), vanadium (V), tellurium (Te), tantalum (Ta), and oxygen (O). The catalyst is represented by the formula MoVTeTaOx. In some embodiments, the catalyst has the formula MoₐV_{b}Te_{c}TadOₓ. In some embodiments, the catalyst has the formula Mo₁V_{0.01-0.3}Te_{0.01-0.9}Ta_{0.01-0.10}, based on the amount of each starting material added to the slurry. In some embodiments, the catalyst has the formula Mo₁V_{0.01-0.3}Te_{0.01-0.9}Ta_{0.01-0.05}. In some embodiments, the catalyst has the formula Mo₁V_{0.39-0.49}Te_{0.1-0.3}Ta_{0.01-0.05}. In some embodiments, the catalyst has the formula Mo₁V_{0.3}Te_{0.05}Ta_{0.05}. In each of these formulations, oxygen is present in sufficient amounts to render the catalyst electrically neutral.

As used herein, the term “catalyst material” refers to a material that includes an active catalyst that can promote the oxidative dehydrogenation of ethane to ethylene, for example, on a carrier. The catalyst material can be a plurality of particles or a formed catalyst material. Non-limiting examples of formed catalyst materials include extruded catalyst materials, pressed (tableted) catalyst materials, compacted/rolled catalyst materials, granular catalyst materials, spherical/spray dried catalyst materials, fluid bed granulators, and cast catalyst materials. Non-limiting examples of pressed and cast catalyst materials includes pellets-such as tablets, ovals, and spherical particles. In some embodiments, binder is used to aid in catalyst forming. In some embodiments, catalyst material formation includes optional workup steps such as: debinding, calcining/sintering, and/or activating/pre-treatment. Workup steps may be introduced to prepare the catalyst to be loaded into a reactor and produce an expected productivity and mitigate any unexpected thermal runaways during startup.

As used herein, the term “catalyst” generally refers to the active catalyst portion, or the active phase, of a catalyst material.

As used herein, the term “chemically compatible carrier” (CCC) refers to a catalyst carrier or support that can be directly added to the hydrothermal synthesis of the catalyst without leading to a substantial reduction of catalyst activity or selectivity, as calculated on the basis of the weight of the active phase. For example, the catalyst material synthesized with a CCC in the hydrothermal synthesis step results in an ethylene selectivity value(S) within about 5% of the ethylene selectivity value of the same catalyst material prepared without carrier, under the same catalyst concentration and reaction conditions. In some embodiments, the catalyst material synthesized with a CCC in the hydrothermal synthesis step results in an ethylene selectivity value(S) at 25% conversion of ethane (T25%) within about 5% of the ethylene selectivity value of the same catalyst material prepared without carrier. In some embodiments, the ethylene selectivity value(S) is within about 2% of the ethylene selectivity value of the same catalyst material prepared without carrier. In some embodiments, the ethylene selectivity value(S) is within about 1.5% of the ethylene selectivity value of the same catalyst material prepared without carrier. In some embodiments, the ethylene selectivity value(S) is within about 1.3% of the ethylene selectivity value of the same catalyst material prepared without carrier. In some embodiments, the CCC is a metal oxide. In some embodiments, the CCC is selected from the group consisting of α-alumina, titania, and WO₃—ZrO₂.

Further, a CCC may refer to a catalyst support or carrier that does not change the hydrothermal reaction manufacturing productivity as compared to the active phase only, which may be observed by preserving the main XRD peaks of the formulated active phase with the presence of a CCC during the hydrothermal synthesis step. The CCC acts as a diluent, so the XRD signal will be weaker than for the active phase only, depending on the amount of CCC added (e.g. 10% carrier corresponds to 10% weaker active phase signal in XRD).

Some carriers, which are not CCCs, may lead to substantial reduction of the catalyst performance, for example, ethylene selectivity and/or ethane conversion, when added during the hydrothermal synthesis. Consequently, not just any carrier can be added during hydrothermal synthesis of the catalyst. The carrier should be selected in a judicious matter based off both short-term and longer-term catalysis performance testing. In some embodiments, there is an emphasis on long-term testing showing no loss of selectivity with time on stream (for example, TOS of >48 hours). As used herein, “time on steam (TOS)” refers to the time the catalyst material spends in the ODH process without interruption.

As used herein, the term “hydrothermal synthesis vessel” refers to a reaction vessel suitable for carrying out a reaction at elevated temperature and pressure, including but not limited to an autoclave, a digestion tank, a pressure vessel, a hydrothermal synthesis reactor, or a PFTE high-pressure tank. In some embodiments, the hydrothermal synthesis vessel is an autoclave.

Conversion of the ethane feed gas was calculated as a volume flow rate change of ethane in the product compared to feed ethane mass flow rate using the following formula:

$$C = ( \frac{2 * {X}_{Ethylene} + {X}_{CO 2} + {X}_{CO}}{2 * {X}_{Ethylene} + 2 * {X}_{Ethane} + {X}_{CO 2} + {X}_{CO}} ) * 100 \%$$

In the above equation, C is the percent of ethane feed gas that has been converted from ethane to another product (i.e., ethane conversion) and X is the molar concentration of the corresponding compound in the gaseous effluent exiting the reactor at corresponding temperature. The ethane conversion was then plotted as a function of temperature to acquire a linear algebraic equation. The linear equation for ethane conversion was solved to determine the temperatures in which the ethane conversion of 35% and 50% were achieved (i.e., 35 and 50% conversion temperatures).

Furthermore, the gas exiting the reactor was analyzed by gas chromatography to determine catalyst or catalyst material selectivity to ethylene (i.e., the percentage on a molar basis of ethane that forms ethylene). Selectivity to ethylene was determined using the following equation:

$${S}_{Ethylene} = ( \frac{2 * {X}_{Ethylene}}{2 * {X}_{Ethylene} + {X}_{CO 2} + {X}_{CO}} ) * 100 \%$$

In the above equation, S_{Ethylene} is the selectivity to ethylene and X is the molar concentration of the corresponding compound in the gaseous effluent exiting the reactor at corresponding temperature. After the 35 and 50% conversion temperature was determined, the above equation for selectivity was solved using the corresponding values for X_{Ethylene}, X_{CO2}, and X_{CO} at the 35% conversion temperature.

GHSV (gas hourly space velocity) is defined as volumetric flow of the reactor feed gas divided by the volume of the catalyst bed. For GHSV values of Catalyst Materials, the catalyst bed is treated as catalyst only (not including carrier) where an assumption was made that the total volume of the catalyst material measured when multiplied by the wt. % of catalyst is the volume of the catalyst. The GHSV was calculated based off the measured volume of the pressed particles (before mixing with quartz sand) and varied depending on each catalyst or catalyst material bulk density. For catalyst materials, the GHSV reported is for the catalyst only, where an assumption was made that the total volume of the catalyst material measured when multiplied by the wt. % of catalyst is the volume of the catalyst.

As used herein, the term “oxidative dehydrogenation” or “ODH” refers to processes that couple the endothermic dehydration of an alkane with the strongly exothermic oxidation of hydrogen as is further described herein. For testing catalysts, the ODH reactions herein are assumed to be referring to the ODH of ethane.

Other than in the operating examples or where otherwise indicated, all numbers or expressions referring to quantities of ingredients, reaction conditions, etc. used in the specification and claims are to be understood as modified in all instances by the term “about”. Accordingly, unless indicated to the contrary, the numerical parameters set forth in the following specification and attached claims are approximations that can vary depending upon the desired properties, which the present disclosure desires to obtain. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical values, however, inherently contain certain errors necessarily resulting from the standard deviation found in their respective testing measurements.

In addition, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

### EXAMPLES

### Carrier Reagents

Table 1 shows the carriers used for the preparation of the samples, including the supplier and/or characteristics of each carrier material.

TABLE 1
Carrier Suppliers and/or Characteristics

| Carrier | Supplier and/or Characteristics |
|---|---|
| α-Al₂O₃ | Alfa Aesar, 99.9% (metal basis) CAS: 1344-28-1, |
| | S_{BET} = 10 m²/g |
| TiO₂ | Reakhim, for optics, S_{BET} = 8 m²/g |
| 18% WO₃—ZrO₂ | Saint-Gobain NorPro, SZ 6*142, S_{BET} = 130 m²/g, |
| | 18% WO₃ |
| SiC | “Russkiy katalizator” > 95% SiC |
| MgAl Spinel | Sasol Mg 63 NT 63.5% MgO, 36.5% Al₂O₃ |
| γ-Al₂O₃ | Haldor Topsoe, VERSAL Alumina VGL 250, S_{BET} = |
| | 250 m²/g |
| Ca-aluminate | CaO - 35 . . . 45 wt. %; Al₂O₃ - 30 . . . 50 wt. %; |
| | Fe₂O₃ - 0 . . . 15 wt. %; SiO₂ - 5 . . . 15 wt. %. |

### Preparation of Samples 1C, 2E, 3E, and 4C, 5E, 6C, and 7C: Hydrothermal Catalyst

### Material Synthesis

### Catalyst Precursors

Commercial anhydrous Ta₂O₅ (CAS 1314-61-0, 99.8% Ta₂O₅) was used as purchased from Plazomotherm. According to the TGA analysis, this sample is thermo-amorphous.

TABLE 2
Conditions for Each Sample

| | | | | | | | | | | Sample |
|---|---|---|---|---|---|---|---|---|---|---|
| | MoO₃ | V₂O₅ | TeO₂ | Ta₂O₅ | CA | OA | EG | Carrier | | Weight |
| Sample¹ | (g) | (g) | (g) | (g)² | (g) | (g) | (g) | (g) | Carrier | (g)³ |
| 1C | 5.3951 | 1.0257 | 0.2985 | 0.4189 | 0.5437 | 0.1680 | 0.1726 | 0.0 | — | 5.6554 |
| 2E | 5.3978 | 1.0255 | 0.3008 | 0.4195 | 0.5410 | 0.1690 | 0.1904 | 15.0318 | α-Al₂O₃ | 20.1528 |
| 3E | 5.4038 | 1.0256 | 0.3023 | 0.4214 | 0.5445 | 0.1712 | 0.1915 | 15.0050 | TiO₂ | 19.9013 |
| 4C | 5.4037 | 1.0277 | 0.2995 | 0.4197 | 0.5419 | 0.1718 | 0.1835 | 15.0044 | γ-Al₂O₃ | 22.7678 |
| 5E | 5.4018 | 1.0230 | 0.2998 | 0.4197 | 0.5461 | 0.1690 | 0.1763 | 15.0013 | 18% WO₃—ZrO₂ | 19.3318 |
| 6C | 5.4051 | 1.0277 | 0.3009 | 0.4193 | 0.5436 | 0.1710 | 0.1823 | 13.5244 | MgAl | 17.8393 |
| | | | | | | | | | Spinel | |
| 7C | 5.4002 | 1.0232 | 0.3010 | 0.4196 | 0.5419 | 0.1708 | 0.1769 | 15.0017 | SiC | 19.3567 |

¹C indicates comparative samples, E indicates experimental samples
²Commercial anhydrous Ta₂O₅ as purchased
³Not calcined, after drying

### General Catalyst Material Synthesis

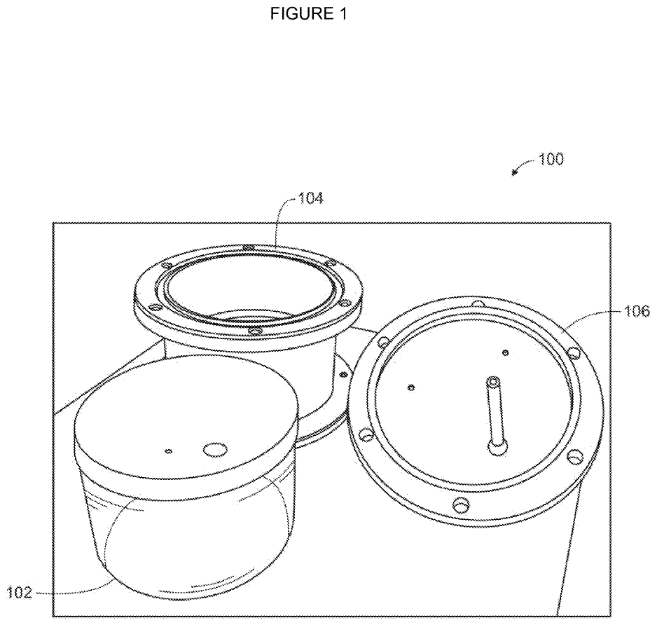

The syntheses of the samples shown in Table 2 were performed hydrothermally. Each sample had a catalyst formula of MoV_{0.30}Te_{0.05}Ta_{0.05}Oₓ. Samples of MoO₃, V₂O₅, TeO₂, Ta₂O₅, citric acid (CA), oxalic acid (OA), and ethylene glycol (EG), in the amounts described above, were dispersed in 200 mL of distilled water at ambient temperature. For samples 2E, 3E, and 4C, 5E, 6C, and 7C, after mixing the main components, approximately 15 g of corresponding carrier (see Table 2 for amounts) was added to the resulting suspension with vigorous stirring of the resulting mixture. For each sample, the slurry was transferred to a stainless-steel autoclave with a glass liner (FIG. 1). The autoclave was immediately sealed without purging the remaining air volume with inert gas. The stirring of the mixture was started simultaneously with the temperature ramp. The syntheses were performed at 190 °C for 48 hours. Once the temperature program was finished, the reacting slurry in the vessel was cooled down to room temperature while maintaining stirring. The solid was separated by filtration, and washed with 2.5 times the volume of distilled water. For samples 2E, 3E, 5E, and 7C, the mother liquor had a characteristic dark blue color due to the presence of vanadyl ion VO₂⁺ in the solution. For samples 4C and 6C, the stock solution was colorless and transparent. The samples obtained were dried in static air at 80 °C overnight.

After synthesis, the catalyst materials were ground into a powder using a mortar/pestle. The catalyst materials were loaded in a quartz boat, and the boat was placed into a quartz furnace tube, which was used for the calcination. The setup was purged with a purified nitrogen flow (<0.3 ppm O₂), and then a heating program on the furnace was started, with a ramp rate from ambient temperature to 600 °C in 6 hours, and then the furnace was held at 600 °C for 2 hours. The calcination proceeded under a slow stream of purified nitrogen, about 30 ml/min, that was vented through a silicone oil bubbler to prevent air infiltration. The solids obtained were ground, pressed into tablets, tablets crushed up into granular particles, and then the resulting particles sieved to collect a fraction of 0.25-0.75 mm resulting in final catalyst materials, which were tested for activity and selectivity.

### Preparation of Samples 8C-14C: Mechanical Mixing

TABLE 3
Conditions for Each Sample 8C-14C

| Sample¹ | Carrier | Sample 1C:Carrier wt.:wt. |
|---|---|---|
| 8C | α-Al₂O₃ | 1:3 |
| 9C² | α-Al₂O₃ | 1:3 |
| 10C | γ-Al₂O₃ | 1:3 |
| 11C | AlO(OH) (boehmite) | 1:3 |
| 12C | Ca-aluminate | 1:3 |
| 13C | TiO₂ | 1:1 |
| 14C | TiO₂ | 1:3 |

¹C indicates comparative sample.
²Prepared with non-calcined Sample 1C precursor (see preparation below).

### Preparation of Samples 8C and 10C-14C

The samples shown in Table 3 were prepared by mechanical mixing of carrier with calcined Sample 1C. Weighted amounts of the two components (see Table 3) were placed in agate mortar and ground by pestle for 10 min. in dry state. Then a few drops of bi-distilled water were added, and the slurry obtained was ground for 10 more minutes. Samples were dried overnight at room temperature, ground, pressed, crushed and sieved to prepare catalyst fraction for testing. No additional calcination of composite samples was done before testing in ODH.

### Preparation of Sample 9C

Sample 9C was prepared by mechanical mixing of the non-calcined Sample 1C precursor with α-Al₂O₃. The mixture was then calcined in pure N₂ at 600 °C for 2 hours.

### Instruments and Measurements

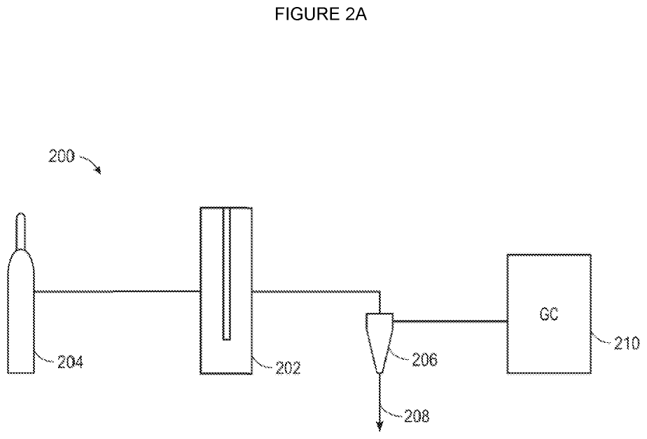

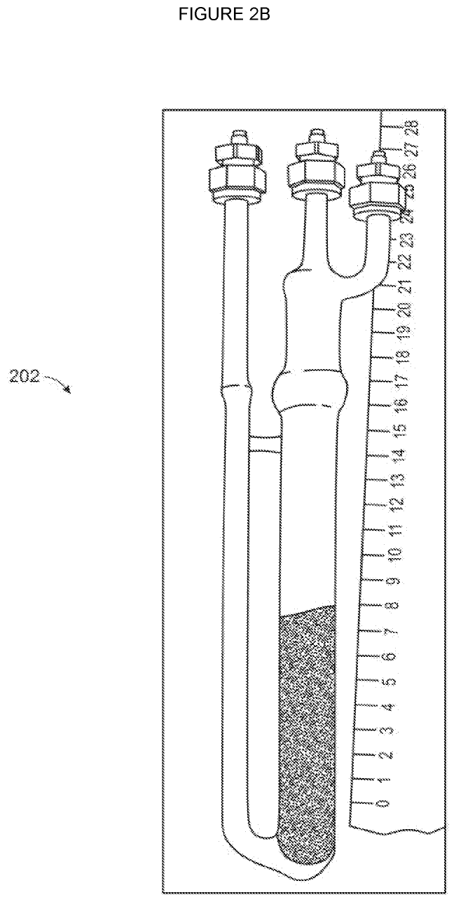

FIGS. 2A and 2B show a test apparatus for testing the ODH performance of the catalyst materials. The catalyst materials were prepared for testing via pressing the calcined catalyst material powder into the shape of rectangular prism. The pressed material was crushed to produce finer particles, and sieved to have the catalyst material particles fraction of 0.25-0.75 mm, which was tested as a representative catalyst material sample via procedure described below.

FIG. 2A is schematic drawing of test apparatus 200 for testing the ODH performance of the catalyst materials. The catalyst materials were tested in ethane oxidation at 320-410 °C by placing the sample charge (0.1 cm³, ~0.15 g, fraction 0.25-0.75 mm) in a fixed-bed quartz reactor 202. The crushed and sieved fraction of each sample was placed into a standard quartz micro-reactor without dilution. Therefore, the catalyst charge contained nearly the same weight amount of the active phase for each sample, facilitating a direct comparison of catalytic data with results obtained for samples synthesized by hydrothermal treatment and mechanical mixing, and for samples with and without added carrier. The weight of catalyst for each test was about 120-140 mg.

A mixed gas feed cylinder 204 was coupled to the fixed-bed quartz reactor 202 through a stainless steel (SS316L) ethane/oxygen (77/23 volume ratio) supply line. The outlet of the fixed-bed quartz reactor 202 was connected to a stainless steel (SS316L) tubing routing the outgoing product gas flow passed through a glass water trap 206 (20 °C) for removal of the excess water and any condensable materials 208. Samples of the reactor effluent were injected periodically into an online chromatograph 210 equipped with the Poropac-Q column and a TCD detector. The catalyst material performance was measured with an accuracy+5%. The connection between the quartz tube and the stainless steel lines ( 1/16″) was done, using Teflon ferrules. The fixed-bed quartz reactor 202 was wrapped with heating tape and placed in an insulation housing. The probe of a thermal couple was placed on the outside of the quartz tube wall where catalyst zone is located.

The flow rate of the ethane/oxygen gas mixture was adjusted to 600 cm³/hour. The reactor was heated to 320 °C with a flow of mixed ethane and oxygen and stabilized for 40 minutes. GC samples were taken periodically in 10 °C to 20 °C steps. For each measurement step, the reactor was stabilized for 40 minutes before injecting the sample to the online GC.

As shown in FIG. 2B, the reactor 202 is a U shaped quartz tube (OD: 6 mm, ID: 4 mm) that was filled with a uniform mixture of equal volume (0.1 cm³ each) of catalyst and quartz particles (0.75-1 mm sizes). The reactor volume that was not occupied by catalyst bed was filled with quartz particles (0.75-1 mm sizes).

The reaction mixtures used for testing was about 78 vol. % ethane and about 22 vol. % oxygen, although some variation around this number, such as 75 vol. % ethane and about 25 vol. % oxygen, do not substantially affect the results. The gas chromatograph 232 was a Chromatek-Kristall 5000 with a thermal conductivity detector, using a column type-M ss316 3M*2 mm, Hayesep Q 80/100 mesh, and a column M ss316 3M*2 mm, NaX 60/80 mesh. The column temperature was ramped according to the following program: 40 °C for 1.5 minutes, then rising to 100 °C at a speed of 15 °C/min. For the corresponding calculations, calibration standards were used to establish response factors.

To accurately identify the resulting products, the exact retention time of each component of the mixture, including reagents and all possible products of this reaction, was determined using the gas chromatograph 210 before conducting the experiment.

### Summary of Catalyst Material Performance

The results on the samples prepared by hydrothermal synthesis with and without carrier, as well as mechanical mixing with carrier, are summarized in Tables 4 and 5.

TABLE 4
Temperatures of 25%-Conversion of Ethane (T_{25%}) and Corresponding
Values of Selectivity of Ethylene Formation (S) for Catalyst
Materials Prepared with and without Al-based Carriers.

| Catalyst | | | |
|---|---|---|---|
| Material | Carrier | T_{25% ethane conv.}, °C | S, % |
| 1C | Pure active phase (no carrier) | 334 | 98.1 |
| 2E | α-Al₂O₃ (hydrothermal synthesis) | 323 | 96.8 |
| 4C | γ-Al₂O₃ (hydrothermal synthesis) | ~390 | 50 |
| 8C | α-Al₂O₃ (mechanical mixture) | 328 | 98.0 |
| 9C | α-Al₂O₃ (mechanical mixture) | 350 | 97.6 |
| 10C | γ-Al₂O₃ (mechanical mixture) | 335 | 96.5 |
| 11C | AlO(OH) (mechanical mixture) | 348 | 97.0 |
| 12C | Ca-aluminate (mechanical mixture) | 370 | 96.0 |

As one can see, sample 2E, prepared by hydrothermal synthesis with α-Al₂O₃ carrier, as compared to sample 1C, prepared without carrier, demonstrates only a minor loss of the selectivity. Accordingly, the preparation of the active phase catalyst precursor is not substantially hindered by the presence of α-alumina in the reaction mixture at the stage of hydrothermal treatment, and subsequent calcination of the composite leads to the formation of the active phase demonstrating quite good catalytic properties.

Sample 4C demonstrates the importance of the selection of a chemically compatible carrier (CCC). Hydrothermal preparation of the same composite with γ-Al₂O₃ resulted in a catastrophic loss of the selectivity. Therefore, the effect caused by two different modifications of alumina (α- and γ-) differs drastically. The results obtained for sample 4C demonstrate a strong negative influence of addition of a carrier. Accordingly, it seems that the addition of γ-Al₂O₃ to the reaction mixture deeply affects the process of formation of the active phase upon hydrothermal synthesis.

Samples 8C-12C show comparative results for the mechanical mixing of the catalyst with various aluminum-based carriers. As shown by sample 8C, no loss of selectivity takes place as a result of dilution of the active phase by α-Al₂O₃. Sample 9C, prepared by mechanical mixing of the non-calcined precursor of sample 1C with α-Al₂O₃ carrier, demonstrates a very slight loss in selectivity. Sample 2E, prepared by hydrothermal synthesis with α-Al₂O₃ carrier, as compared to samples 8C and 9C, prepared by mechanical mixing with α-Al₂O₃ carrier, demonstrates only a minor loss of the selectivity. Accordingly, the preparation of the active phase catalyst precursor is not substantially hindered by the presence of α-alumina in the reaction mixture at the stage of hydrothermal treatment.

Sample 10C shows that even mechanical mixing of the active phase with γ-Al₂O₃ causes a measureable loss of the activity and selectivity of the resulting catalyst material.

Samples 11C and 12C demonstrate results for mechanical mixing of catalyst sample 1C with other alumina sources. Sample 11C, prepared by mechanical mixing with AlO (OH) (boehmite) demonstrated a minor loss of activity and selectivity. Sample 12C, prepared by mechanical mixing with calcium aluminate, showed substantial suppression of the catalyst activity and selectivity, especially at a high O₂ conversion.

TABLE 5
Temperatures of 25%-Conversion of Ethane (T_{25%}) and Corresponding
Values of Selectivity of Ethylene Formation (S) for Catalyst Materials
Prepared with and without TiO₂.

| Catalyst | | T_{25% ethane conv.}, | |
|---|---|---|---|
| Material | Carrier | °C | S, % |
| 1C | Pure active phase (no carrier) | 334 | 98.1 |
| 3E | TiO₂ (hydrothermal synthesis) | 337 | 97.0 |
| 13C | TiO₂ (1:1 mechanical mixture | 329 | 98.0 |
| | 1C:TiO₂) | | |
| 14C | TiO₂ (1:3 mechanical mixture | 327 | 98.0 |
| | 1C:TiO₂) | | |

As one can see, sample 3E, prepared by hydrothermal synthesis with TiO₂ carrier, as compared to sample 1C, prepared without carrier, demonstrates only a minor loss of the selectivity. Accordingly, the preparation of the active phase catalyst precursor is not substantially hindered by the presence of titania in the reaction mixture at the stage of hydrothermal treatment, and subsequent calcination of the composite leads to the formation of the active phase demonstrating quite good catalytic properties.

Samples 13C and 14C show comparative results for the mechanical mixing of the catalyst sample 1C with titania in 1:1 and 1:3 ratios, respectively. As shown by sample 13C and 14C, no loss of selectivity takes place as a result of dilution of the active phase by titania. Sample 3E, prepared by hydrothermal synthesis with titania, as compared to samples 13C and 14C, prepared by mechanical mixing of catalyst with TiO₂, demonstrates only a minor loss of the selectivity. Accordingly, the preparation of the active phase catalyst precursor is not substantially hindered by the presence of titania in the reaction mixture at the stage of hydrothermal treatment.

### Discussion of Results for Individual Catalyst Materials

### Sample 1C (Pure Active Phase):

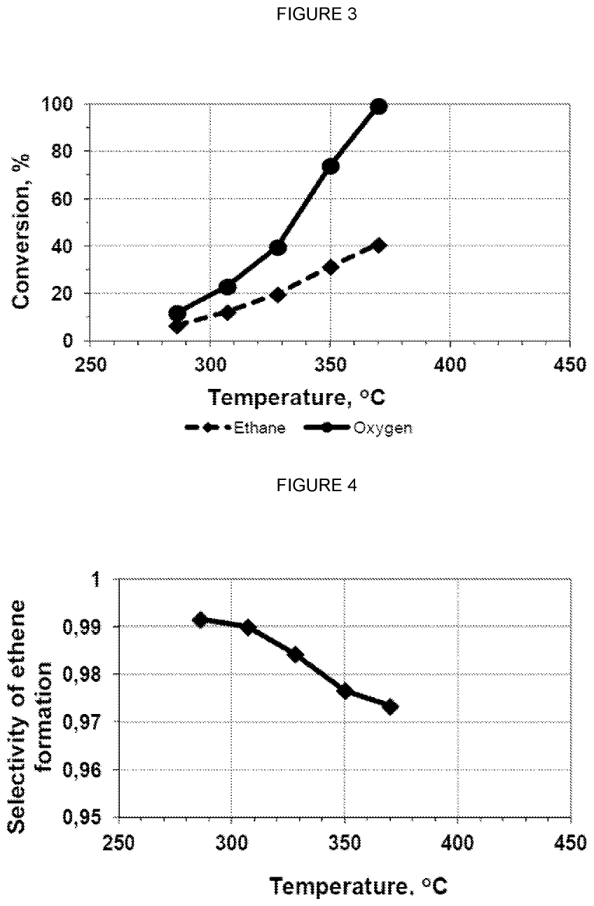

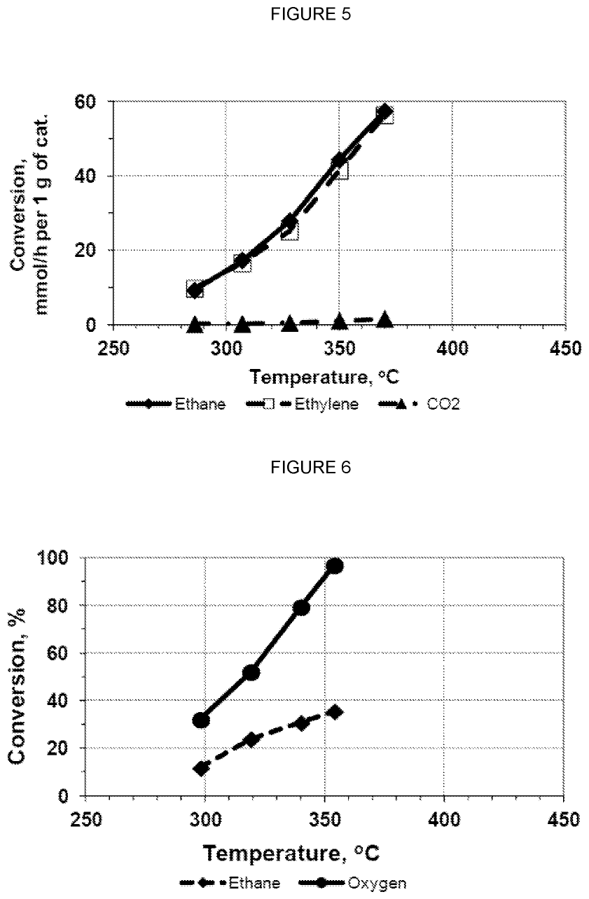

FIG. 3 is a plot of the conversion of reagents to ethylene for comparative sample 1C. FIG. 4 is a plot of the selectivity of ethylene formation for sample 1C. FIG. 5 is a plot of the conversion/space yields for sample 1C. The tests were run using 0.138 g of catalyst with a gas mixture of 78 vol. % ethane and 22 vol. % oxygen, at a flow rate of 600 cm³/h and a GHSV=3000 h⁻¹.

Sample 2E (Hydrothermal Synthesis with α-Alumina):

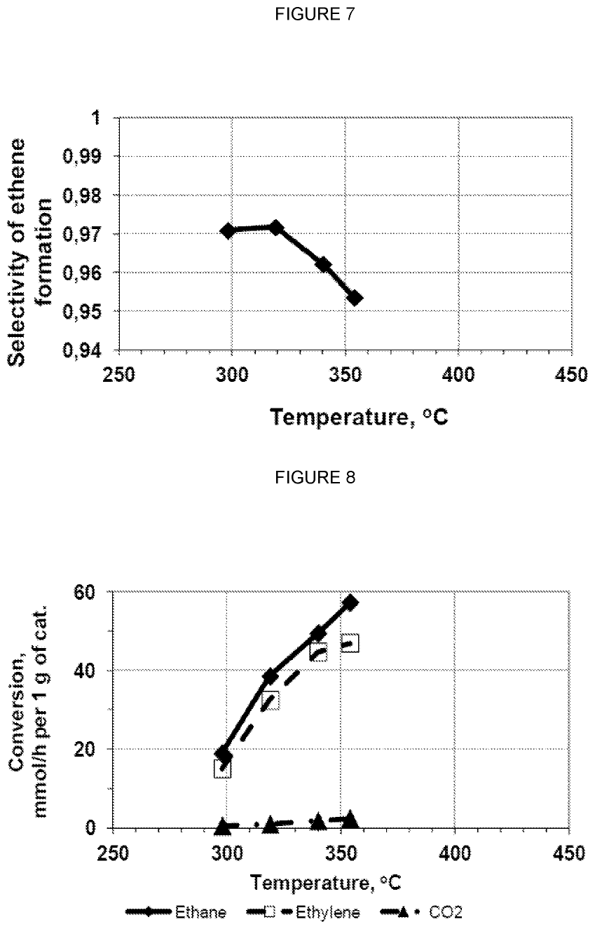

FIG. 6 is a plot of the conversion of ethylene formation for sample 2E. FIG. 7 is a plot of the selectivity of ethylene formation for sample 2E. FIG. 8 is a plot of the conversion/space yields for sample 2E. The tests were run with about 0.121 g of the active phase using a gas mixture of 78 vol. % ethane and 22 vol. % oxygen at a flow rate of 600 cm³/h and a GHSV=1500 h⁻¹.

As shown in FIGS. 6-8, synthesis of the active phase was not substantially affected by the presence of α-alumina in the reaction mixture at the stage of hydrothermal treatment. Subsequent calcination of the composite led to the formation of a catalyst material where the active phase demonstrated quite good catalytic properties, which are comparable to the active phase only as shown in FIGS. 3-5. This demonstrates that there not a substantial negative impact of α-alumina in the hydrothermal synthesis step.

Sample 3E (Hydrothermal Synthesis with Titania):

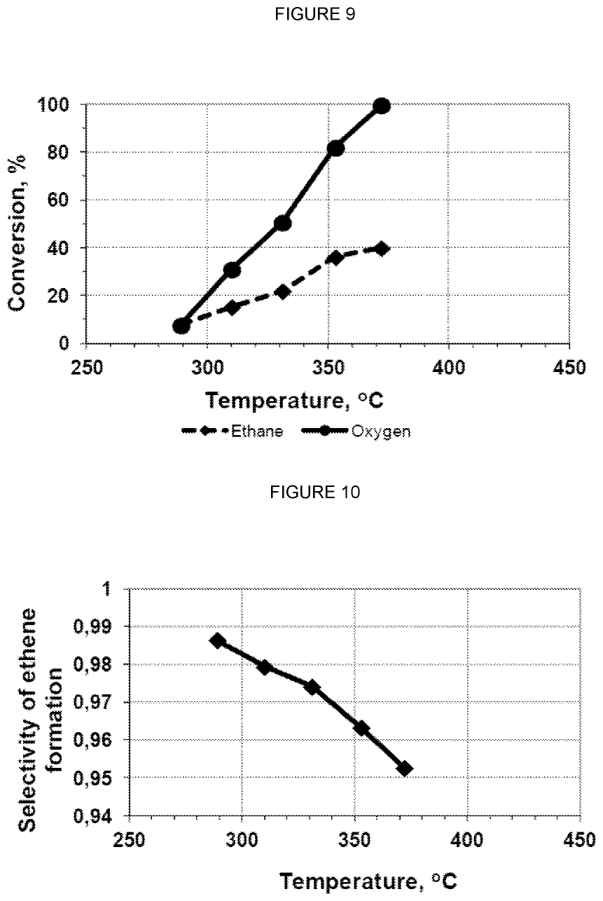

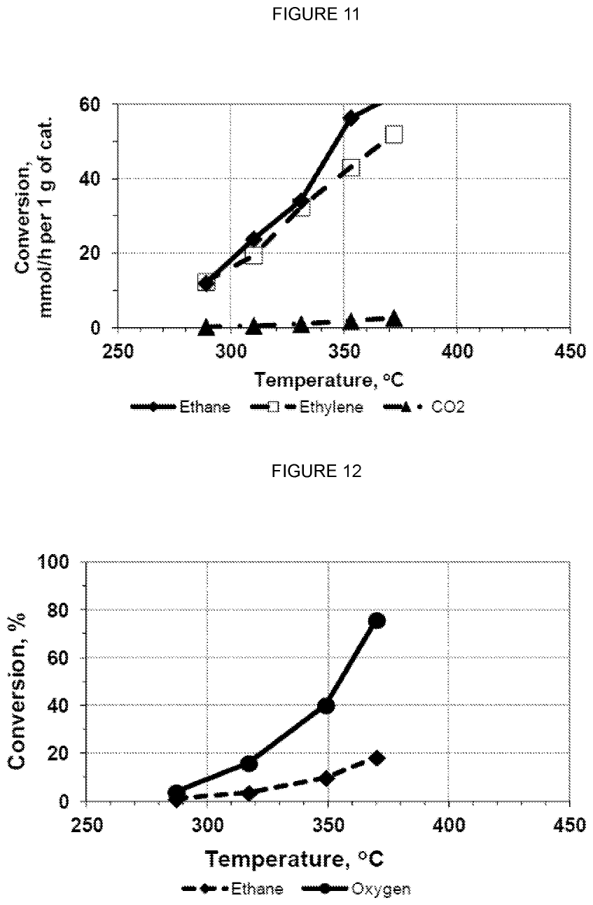

FIG. 9 is a plot of the conversion of ethylene formation for sample 3E. FIG. 10 is a plot of the selectivity of ethylene formation for sample 3E. FIG. 11 is a plot of the conversion/space yields for sample 3E. The tests were run on about 0.125 g of the active phase using a gas mixture of 78 vol. % ethane and 22 vol. % oxygen at a flow rate of 600 cm³/h and a GHSV=1500 h⁻¹.

As shown in FIGS. 9-11, performance of the synthesized catalyst material was not substantially affected by the presence of titania in the reaction mixture at the stage of hydrothermal treatment. When comparing the catalytic curves of FIGS. 9-11 with that of the active phase only shown in FIGS. 3-5, only a small loss of the selectivity is observed. This demonstrates that including titania in the hydrothermal synthesis step does not have a substantial negative impact on the catalyst.

Sample 4C (Hydrothermal Synthesis with γ-Alumina):

A comparative sample using γ-Al₂O₃ with a high surface area (~250 m²/g) was prepared by hydrothermal synthesis to check the role of modification of pure alumina.

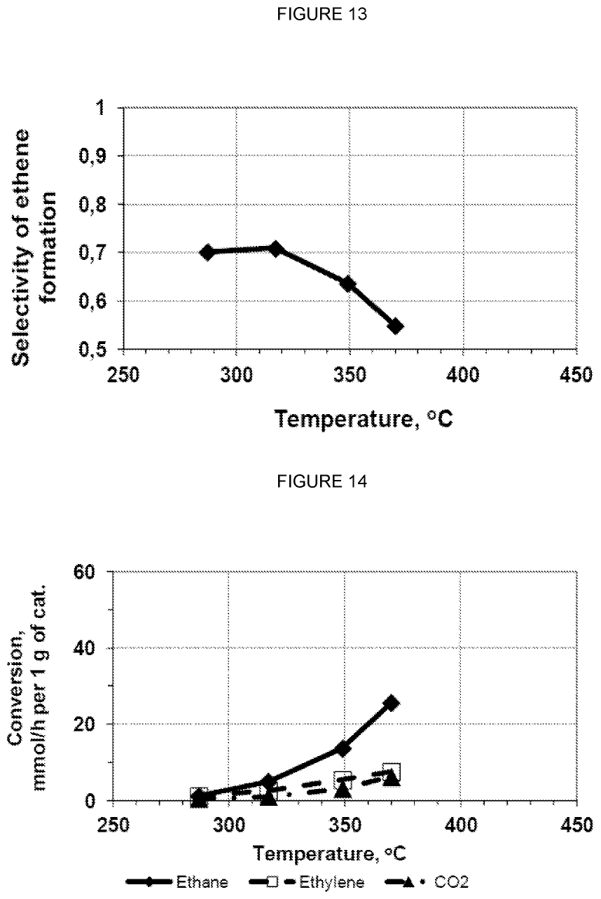

FIG. 12 is a plot of the conversion of ethylene formation for sample 4C. FIG. 13 is a plot of the selectivity of ethylene formation for sample 4C. FIG. 14 is a plot of the conversion/space yields for sample 4C. The tests were run on about 0.140 g of the active phase using a gas mixture of 78 vol. % ethane and 22 vol. % oxygen at a flow rate of 600 cm³/h and a GHSV=1000 h⁻¹.

The addition of γ-alumina during the hydrothermal synthesis step negatively affected the performance of the catalyst. FIGS. 12-14 show a drastic decline in the catalyst material performance of sample 4C as compared to the active phase only (sample 1C) shown in FIGS. 3-5. The addition of γ-alumina in the hydrothermal synthesis step results in significant decline in the selectivity of ethylene (see FIG. 13), as compared to sample 1C (see FIG. 4). This clearly demonstrates the incompatibility of γ-alumina with the catalyst and the substantial negative affect of the addition of γ-alumina during the hydrothermal synthesis.

Sample 5E (Hydrothermal Synthesis with 18% WO₃-ZrQ₂:

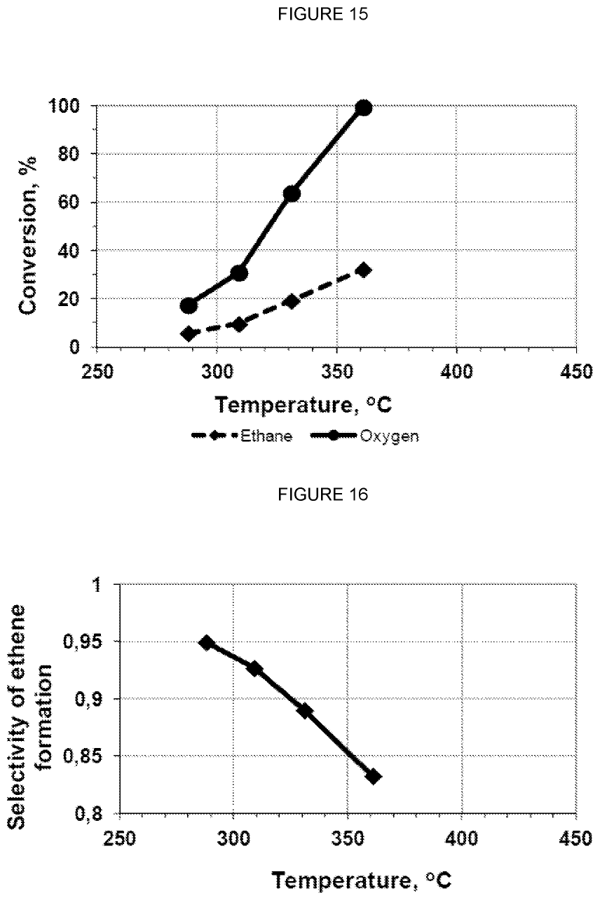

Sample 5E was prepared hydrothermally with 18% WO₃—ZrO₂. FIG. 15 is a plot of the conversion of ethylene formation for sample 5E. FIG. 16 is a plot of the selectivity of ethylene formation for sample 5E. The tests were run on about 0.124 g of the active phase using a gas mixture of 78 vol. % ethane and 22 vol. % oxygen at a flow rate of 600 cm³/h and a GHSV=1500 h⁻¹.

The results obtained for sample 5E illustrate that the WO₃—ZrO₂ carrier did not significantly adversely affect the catalyst. Although sample 5E demonstrates a loss of the selectivity in ODH compared to Sample 1C (FIG. 16), sample 5E showed high activity (FIG. 15).

Sample 6C (Hydrothermal Synthesis with Spinel 63.5% MgQ-Al₂O₃):

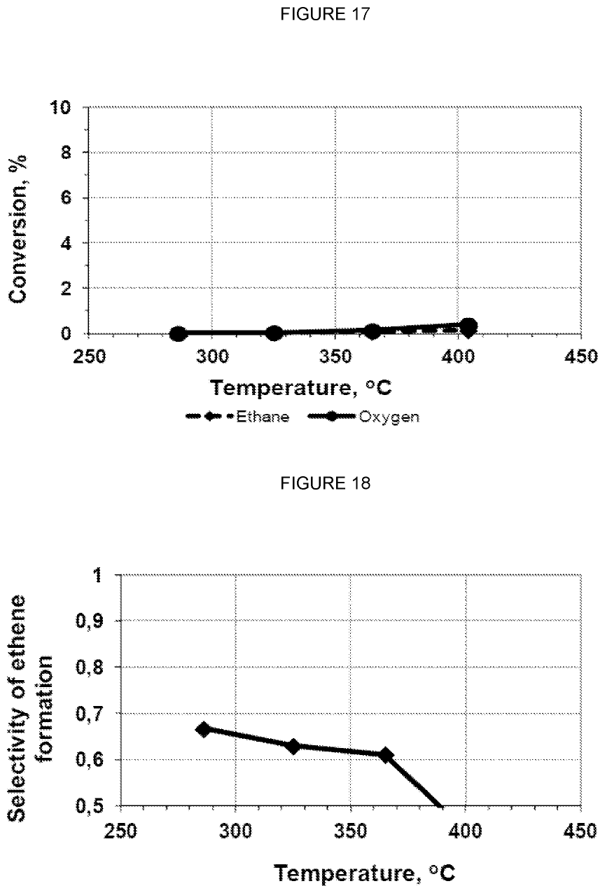

FIG. 17 is a plot of the conversion of ethylene formation for sample 6C. FIG. 18 is a plot of the selectivity of ethylene formation for sample 6C. The tests were run on about 0.105 g of the active phase using a gas mixture of 78 vol. % ethane and 22 vol. % oxygen at a flow rate of 600 cm³/h and a GHSV=1500 h⁻¹.

The use of the 63.5% MgO—Al₂O₃ spinel as a carrier added in hydrothermal synthesis causes a complete loss of the catalyst activity in both ODH and complete oxidation reactions. Sample 6C demonstrates negligible activity in both reactions (FIGS. 17-18). It appears that the added 63.5% MgO—Al₂O₃ completely suppresses the formation of the quaternary oxide precursor Mo₁—V_{0.3}—Te_{0.05}—Ta_{0.05}—Oₓ upon hydrothermal synthesis.

Sample 7C (Hydrothermal Synthesis with SiC):

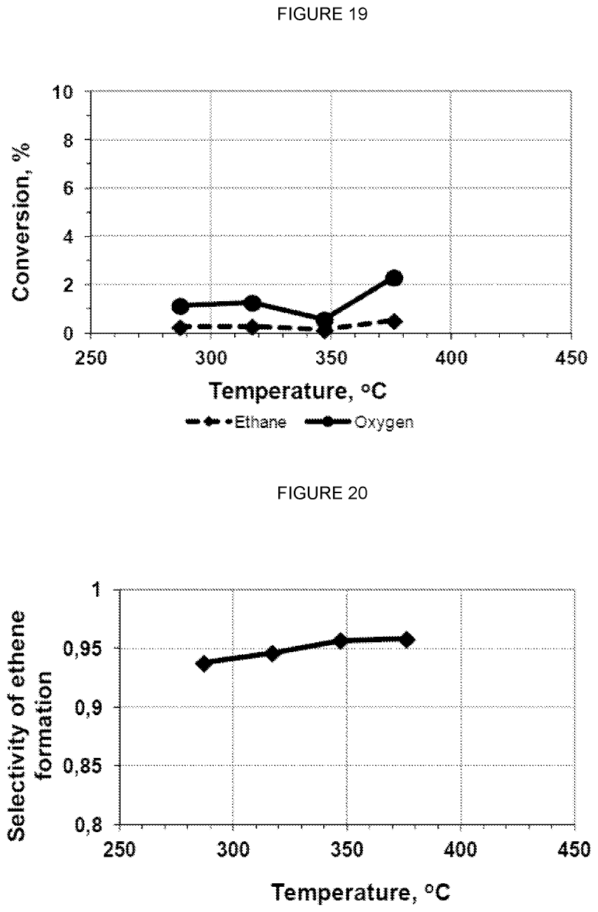

FIG. 19 is a plot of the conversion of ethylene formation for sample 7C. FIG. 20 is a plot of the selectivity of ethylene formation for sample 7C. The tests were run on about 0.12 g of the active phase using a gas mixture of 78 vol. % ethane and 22 vol. % oxygen at a flow rate of 600 cm³/h and a GHSV=1500 h⁻¹.

Complete suppression of the oxidative activity was detected with silicon carbide added as a carrier in the hydrothermal synthesis of the catalyst material.

### Powder X-ray Diffraction (XRD) Characterization

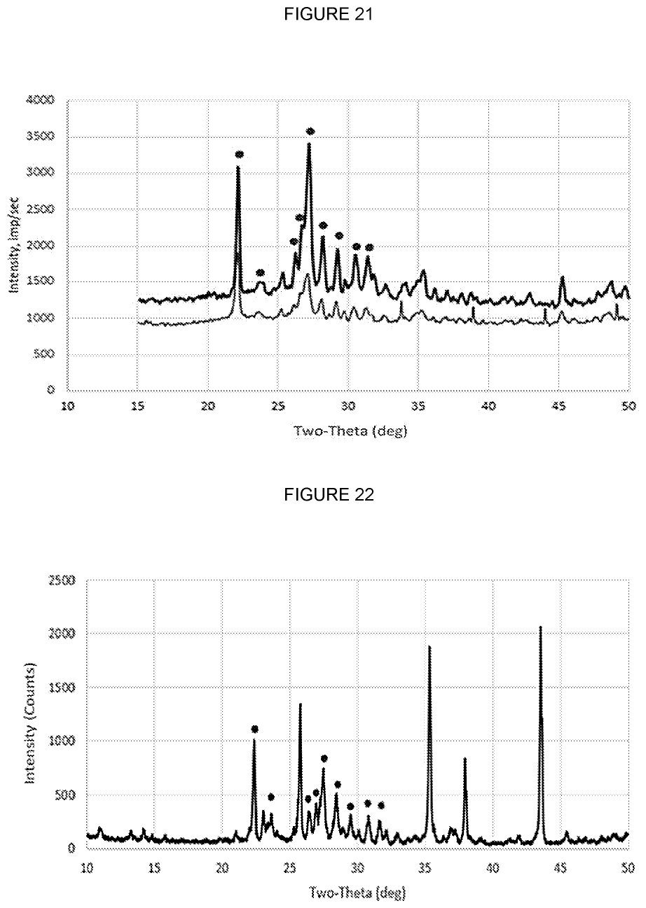

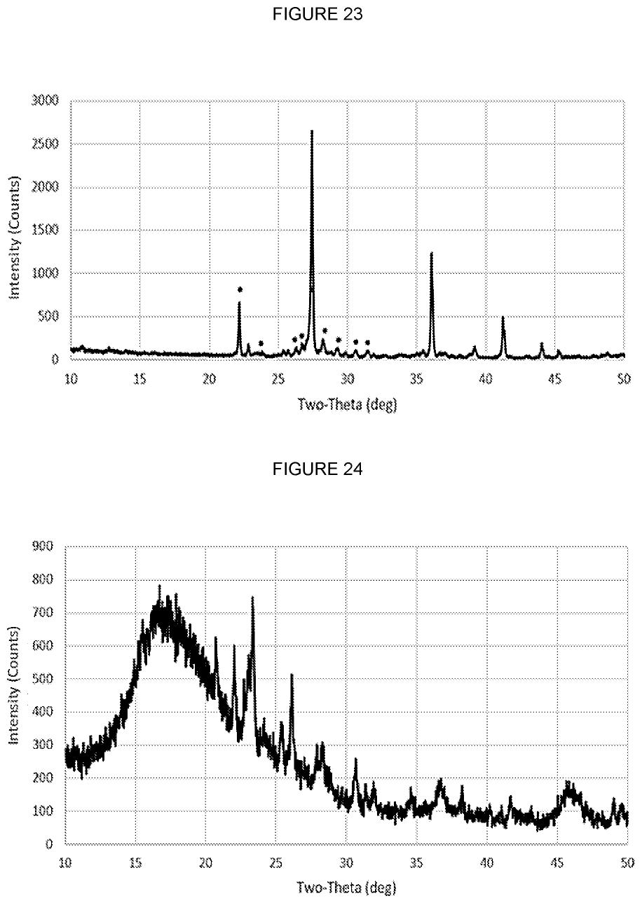

FIG. 21 is an XRD graph of comparative sample 1C, with the darker upper line showing the noncalcined precursor and the lighter lower line showing the calcined catalyst. The dots mark the main peaks. FIG. 22 is an XRD graph of sample 2E, prepared hydrothermally with α-alumina, with dots marking the main peaks of sample 1C. FIG. 23 is an XRD graph of sample 3E, prepared hydrothermally with titania, with dots marking the main peaks of sample 1C. FIG. 24 is an XRD graph of comparative sample 4C, prepared hydrothermally with γ-alumina. XRD for samples 1C, 2E, 3E, and 4C was run on the same instrument under the same conditions. Powder X-ray Diffraction patterns were collected using an instrument with a monochromated Cu Kα source (2=1.5406 Å). Patterns were collected from 10-60° 20 with a step size of 0.02° 20 scan rate of 0.02°20/s.

As one can see in FIGS. 22 and 23, the main peaks of catalyst sample 1C, prepared without carrier, are preserved for samples 2E and 3E, prepared hydrothermally with a CCC.

Other implementations are also within the scope of the following claims.

1. A method for preparing a catalyst material comprising:
forming a slurry comprising oxides of molybdenum, tantalum, vanadium, and tellurium;
adding citric acid, oxalic acid, and ethylene glycol to the slurry;
adding a chemically compatible carrier (CCC) to the slurry;
transferring the slurry to a hydrothermal synthesis vessel;
heating the hydrothermal synthesis vessel to form a catalyst material precursor;
isolating the catalyst material precursor formed in the hydrothermal synthesis vessel; and
calcining the catalyst material precursor to form the catalyst material.

2. The method of claim 1, wherein the CCC comprises α-alumina, titania, or WO₃—ZrO₂.

3. The method of claim 1, wherein the CCC is α-alumina.

4. The method of claim 1, wherein the CCC is titania.

5. The method of claim 1, wherein the catalyst material has an ethylene selectivity at 25% ethane conversion within about 5% of the ethylene selectivity of the same catalyst material prepared without the CCC.

6. The method of claim 1, comprising using anhydrous tantalum oxide as the oxide of tantalum in the slurry.

7. The method of claim 1, comprising heating the hydrothermal synthesis vessel at 190 °C for 48 hours.

8. The method of claim 1, comprising calcining the catalyst material precursor by: placing the catalyst material precursor in a furnace under a purified nitrogen atmosphere; ramping a temperature of the furnace from ambient to a temperature of 600 °C over 6 hours; and holding the temperature at 600 °C for 2 hours.

9. The method of claim 1, wherein the catalyst material comprises a catalyst comprising the formula:
MoₐV_{b}Te_{c}Ta_{d}Oₓ
wherein:
a is 1.0;
b is about 0.01 to about 0.3;
c is about 0.01 to about 0.09;
d is about 0.01 to about 0.10; and
x is the number of oxygen atoms necessary to render the catalyst electronically neutral.

10. The method of claim 9, wherein:
b is about 0.2 to about 0.4;
c is about 0.03 to about 0.07; and
d is about 0.03 to about 0.05.

11. The method of claim 9, wherein the catalyst has the formula:
Mo₁V_{0.3}Te_{0.05}Ta_{0.05}Oₓ.

12. The method of claim 11, wherein the catalyst material has a temperature of 25% ethane conversion of from about 323 °C to about 337 °C and a selectivity at 25% ethane conversion of about 97%.

13. A process for oxidative dehydrogenation of ethane, the process comprising contacting a gaseous feed comprising ethane and oxygen with a catalyst material in a reactor to produce an effluent comprising ethylene, wherein the catalyst material comprises a catalyst and a chemically compatible carrier (CCC), wherein catalyst has the formula:
MoₐV_{b}Te_{c}Ta_{d}Oₓ
wherein:
a is 1.0;
b is about 0.01 to about 0.3;
c is about 0.01 to about 0.09;
d is about 0.01 to about 0.10; and
x is the number of oxygen atoms necessary to render the catalyst electronically neutral, wherein the catalyst material is prepared by:
forming a slurry comprising oxides of molybdenum, tantalum, vanadium, and tellurium;
adding citric acid, oxalic acid, and ethylene glycol to the slurry;
adding a chemically compatible carrier (CCC) to the slurry;
transferring the slurry to a hydrothermal synthesis vessel;
heating the hydrothermal synthesis vessel to form a catalyst material precursor;
isolating the catalyst material precursor formed in the hydrothermal synthesis vessel; and
calcining the catalyst material precursor to form the catalyst material.

14. The process of claim 13, wherein the CCC comprises α-alumina, titania, or WO₃—ZrO₂.

15. The process of claim 13, wherein the CCC is α-alumina.

16. The process of claim 13, wherein the CCC is titania.

17. The process of claim 13, wherein the catalyst material has a selectivity at 25% ethane conversion within about 5% of the selectivity of the same catalyst material prepared without the CCC.

18. The process of claim 13, wherein:
b is about 0.2 to about 0.4;
c is about 0.03 to about 0.07; and
d is about 0.03 to about 0.05.

19. The process of claim 12, wherein the catalyst has the formula:
Mo₁V_{0.3}Te_{0.05}Ta_{0.05}Oₓ.

20. The process of claim 13, wherein the catalyst material has a temperature of 25% ethane conversion of from about 323 °C to about 337 °C and a selectivity at 25% ethane conversion of about 97%.
